(19) United States
(12) Patent Application Publication
(10) Pub. No.: US 2005/0107821 A1
(43) Pub. Date: May 19, 2005

(54) Balloon catheter with kink resistant distal segment

(76) Inventors: John F. Shanley, Redwood City, CA (US); Beau M. Fisher, Danville, CA (US)

Correspondence Address: CINDY A. LYNCH;CONOR MEDSYSTEMS, INC., 1003 HAMILTON COURT, MENLO PARK, CA, 94025, US

(21) Appl. No.: 10/989,954

(22) Filed: Nov. 15, 2004

Related U.S. Application Data
(60) Provisional application No. 60/520,204, filed on Nov. 14, 2003.

(51) Int. Cl. A61M 29/00

(52) U.S. Cl. 606/194; 604/103.04

(57) ABSTRACT

A rapid exchange balloon catheter has a kink resistant distal catheter segment just proximal of the balloon with a kink resisting member to provide pushability and kink resistance while maintaining flexibility. The kink resistant distal segment allows the rapid exchange guidewire tube to be shortened for a more rapid guidewire exchange.

### BACKGROUND

Rapid exchange balloon catheters are described in U.S. Pat. Nos. 4,762,129 and 5,040,548 which are incorporated herein by reference. These rapid exchange catheters include a distal guidewire lumen which extends through the balloon from a distal end of the balloon to a guidewire exit port proximal of the balloon. In these balloon catheter systems a flexible portion of the catheter proximal of the balloon is an important region which is prone to kinking. This portion of the catheter proximal of the balloon and distal to a stiffer proximal catheter section should be simultaneously very flexible to navigate the coronary arteries, have good column strength to provide pushability, and have good kink resistance.

Rapid exchange catheters have the advantage that the guidewire passes only through a short segment at the distal end of the catheter. This greatly decreases the time required to exchange catheters compared to an over the wire catheter in which the guidewire is inserted through a lumen extending the length of the catheter. However, rapid exchange catheters with very short guidewire lumens can be prone to kinking at a location close to the proximal end of the guidewire lumen. Thus, it would be desirable to provide an improved rapid exchange catheter with a short guidewire tube.

### SUMMARY OF THE INVENTION

The present invention relates to rapid exchange balloon catheter having a kink resistant distal segment just proximal of the balloon with a kink resisting member to provide pushability and kink resistance while maintaining flexibility.

In accordance with one aspect of the invention, a balloon catheter comprises a balloon segment having an expandable balloon and a guidewire tube extending through the balloon, the guidewire tube having a proximal port adjacent a proximal end of the balloon, a distal segment connected to a proximal end of the balloon segment and having an inflation lumen and a kink resisting member extending along the length of the distal segment, and a proximal segment having flexibility which is less than a flexibility of the distal segment, the proximal segment connected to a proximal end of the distal segment with the kink resisting member extending into or fixed to the proximal segment.

In accordance with another aspect of the invention a rapid exchange catheter system comprises a balloon catheter and a guidewire. The balloon catheter comprises a balloon segment having an expandable balloon and a guidewire tube extending through the balloon, the guidewire tube having a proximal port adjacent a proximal end of the balloon, a distal segment connected to a proximal end of the balloon segment and having an inflation lumen and a kink resisting member extending along the length of the distal segment, a proximal segment having flexibility which is less than a flexibility of the distal segment, the proximal segment connected to a proximal end of the distal segment with the kink resisting member extending into or fixed to the proximal segment. The balloon segment is fused to the distal segment at a junction area adjacent the proximal port of the guidewire tube. The guidewire is slidably positioned in the guidewire tube.

### BRIEF DESCRIPTION OF THE DRAWINGS

The invention will now be described in greater detail with reference to the preferred embodiments illustrated in the accompanying drawings, in which like elements bear like reference numerals, and wherein:

FIG. 1 is a side view of a rapid exchange balloon catheter according to one embodiment.

FIG. 2 is a cross sectional view, taken along line 2-2 of FIG. 1, of one example of a catheter with a core wire kink resisting member.

FIG. 3 is a side cross sectional view of a portion of one embodiment of the catheter of FIG. 2 with an end sleeve for retaining the core wire distal end.

FIG. 4 is a cross sectional view, taken along line 2-2 of FIG. 1, of an example of a catheter with a combined wire and extrusion kink resisting member which is coextruded with the catheter.

FIG. 5 is a cross sectional view, taken along line 2-2 of FIG. 1 of an example of a catheter with an extruded kink resisting member.

FIG. 6 is a cross sectional view, taken along line 2-2 of FIG. 1 of an example of a catheter with a combined wire and extrusion kink resisting member.

FIG. 7 is a perspective view of one example of the kink resisting member of FIG. 6.

FIG. 8 is a cross sectional view, taken along line 2-2 of FIG. 1 of an example of a catheter with a combined wire and extrusion kink resisting member.

FIG. 9 is a cross sectional view, taken along line 2-2 of FIG. 1 of an example of a catheter with a coiled wire kink resisting member.

### DETAILED DESCRIPTION

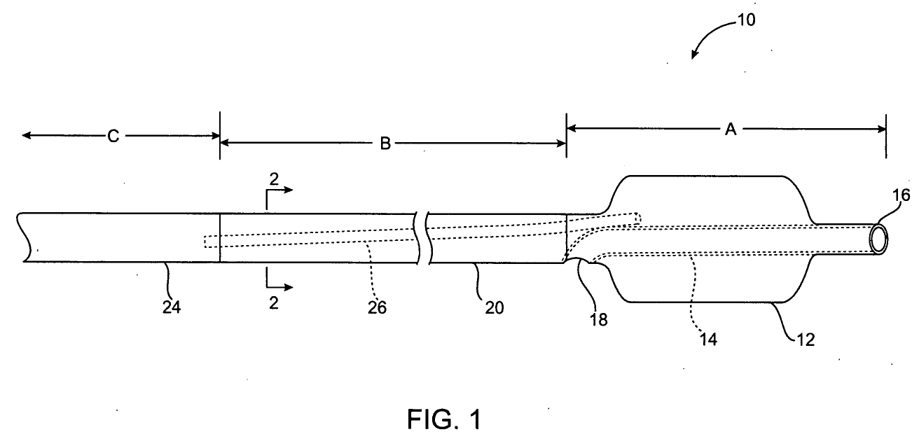

The dilation catheter 10 of FIG. 1 includes a balloon segment A, a distal segment B, and a proximal segment C. The balloon segment A includes a balloon 12 which is shown in an expanded condition in FIG. 1. Within the balloon 12 is a guidewire tube 14 extending from a distal port 16 to a proximal port 18. The guidewire tube 14 extends a very short distance proximally of the balloon with the proximal port 18 of the guidewire tube 14 being adjacent to or close to the balloon to improve the speed and ease of catheter exchanges.

The dilation catheter 10 shown herein is designed as an angioplasty catheter or for delivery of a coronary stent. The stent can be a fully balloon expandable stent or a partially balloon expandable stent. The stent can also be permanent or biodegradable. In addition to use for angioplasty or stent deployment, the balloon catheter can also be used for other known purposes.

The balloon 12 may be formed by any known method, such as by elongating and inflating a tube within a balloon shaped mold. The balloon segment A is connected to the distal segment B by fusing or other means at a junction area which is at or adjacent the proximal port 18 of the guidewire tube. The guidewire tube 14 is fused to the balloon 12 at the distal end and is fused to the balloon and/or the distal segment B at the proximal end of the guidewire tube. The bonding or junction area at which the guidewire tube 14, balloon segment A and distal segment B are fused together are generally formed in a single forming process, such a thermal bonding.

The drawings have illustrated the bonds between the different polymer materials used in the catheter as fused together along a line. In most cases the bonds will be formed by thermal welding and will actually appear as smooth transitions in which the materials are mixed at the junction area.

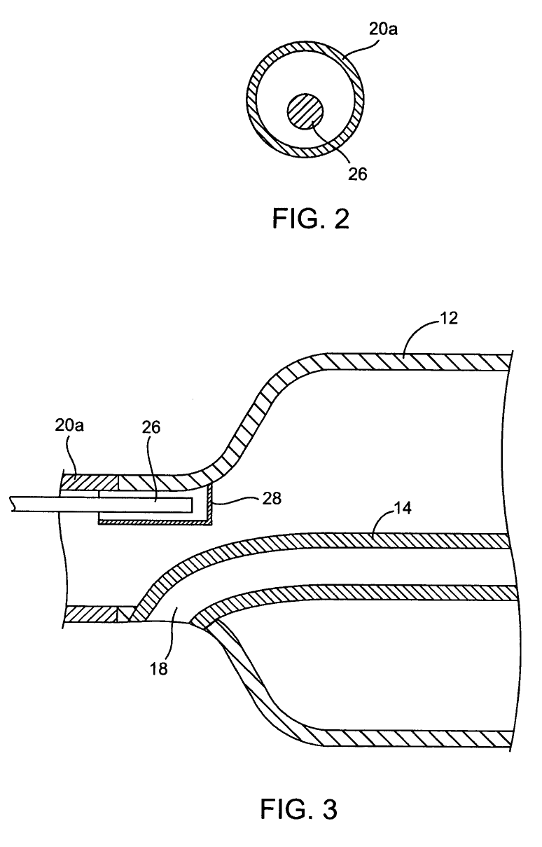

The distal segment B of the catheter 10 is formed by a highly flexible tube 20 which is connected to the balloon segment A and provides an inflation lumen for inflation and deflation of the balloon. The distal segment B may be formed of polymer, a layered arrangement of one or more polymers with one or more coils or braids, or the like. The distal segment B is strengthened by a kink resisting member which may be any of the kink resisting members which will be described herein with reference to FIGS. 2-9. The distal segment B of the catheter 10 uses the kink resisting members described below to provide a kink resistant catheter segment which is designed to provide pushability and kink resistance while maintaining flexibility.

The proximal segment C is formed of a tube 24 of a more rigid material than the distal segment B. For example, the proximal segment may be a metal hypotube, a tube of other metal material, a polymer shaft with metal coils or braids, or the like. In one embodiment, the proximal segment is a stainless steel hypotube. The pushability of the proximal segment C is more important than flexibility since this portion of the catheter will remain within a guide catheter along a path from the femoral artery access site to the vicinity of the heart along a path which is not particularly tortuous. The distal end of the proximal segment C is connected to the distal segment B by any of the known methods, such as epoxy, fusing, or necking a polymer sleeve over the metal and fusing the polymer of segment B to the polymer sleeve. A proximal end of the proximal segment C is connected to a luer fitting or other filling for connection to a source of pressurized fluid for inflation of the balloon.

According to one example, the proximal segment C has a length of at least 50 cm, preferably about 75 cm to about 125 cm. In this example, the distal segment B has a length of at least 5 cm, preferably about 8 cm to about 30 cm. A length of the balloon segment A may be varied depending on a length of a stent to be delivered with the balloon.

FIG. 2 illustrates a distal tube 20a with a kink resisting member in the form of a core wire 26. The core wire 26, as shown in FIG. 1, is bonded to the proximal tube 24 and extends through the distal tube 20a to or into the balloon segment A. Thus, the core wire 26 provides resistance to kinking along the entire length of the flexible distal segment B and particularly and the likely kink points in the transition regions at both ends of the distal segment.

The core wire 26 can be free floating within a lumen of the distal tube 20a providing increased flexibility. Alternatively, to provide additional pushability or column strength, the core wire 26 can be fused to the catheter at one or more points along its length. The core wire 26 may also be extruded within the wall of distal tube 20a. The core wire 26 is preferably a metal or other wire with high column strength, for example, stainless steel, titanium, metal alloys, or shape memory alloys, such as Nitinol. The core wire 26 may be of a constant diameter or of a tapered shape to increase flexibility distally.

The core wire 26 of FIG. 2 can have a diameter such that the core wire itself can provide a mechanical stop which prevents kinking by preventing the tube 20a from collapsing to an oval cross section at which kinking occurs.

FIG. 3 illustrates a core wire 26 which is movable within an end sleeve 28 to provide the improved flexibility of a free floating core wire in combination with the column strength provided when the core wire contacts an end of the sleeve. The end sleeve 28 for the core wire may be bonded to the distal tube 20a, to the balloon 12, or to the guidewire tube 14 in the vicinity of the transition between the distal segment B and the balloon segment A.

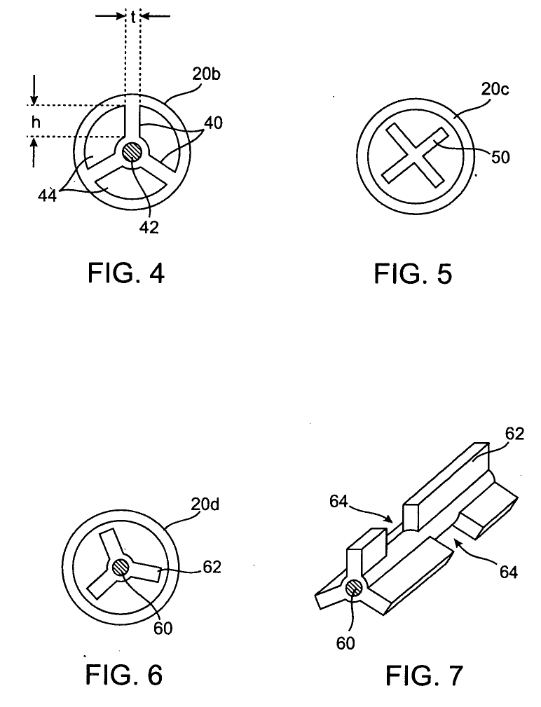

FIG. 4 illustrates another embodiment of a distal segment B having a tube 20b which is extruded with a wagon wheel structure having spokes 40. The wagon wheel spokes 40 and tube 20b are formed of a flexible polymer material which is extruded onto a central wire 42 which together form a kink resisting structure. Alternately, the polymer structure can be extruded and the central wire 42 can be inserted into a central lumen of the extrusion. The central wire 42 and spokes 40 provide a symmetrical structure for uniform bending, prevention of kinking due to the wire and spokes, along with flexibility due to the small diameter of the wire, and pushability provided by the column strength of the wire.

The spokes 40 have a height h and a thickness t. In one embodiment, the height to thickness ratio of the spokes 40 is kept below the critical buckling ratio in the radial direction. Thus, the spokes will compress rather than buckling and will resist bucking of the catheter. Although three spokes 40 have been shown, other numbers may also be used.

The three fluid transmitting chambers 44 of the FIG. 4 embodiment can each transmit fluid independently, or can be provided with openings between the chambers for cross fluid flow.

In one example, the wire 42 of FIG. 4 is a Nitinol core wire which provides very good column strength and pushability combined with the flexibility of the pseudo elastic range of the Nitinol. Due to the asymmetry of the stress strain curve of Nitinol in the 1^{st} and 3^{rd} quadrants (in tension and in compression), Nitinol provides a column strength similar to stainless steel with a flexibility greater than stainless steel in the pseudo elastic range.

As in the core wire embodiments of FIGS. 1-3, the core wire 42 of the FIG. 4 embodiment preferably extends to or just past the transition from the distal segment B to the proximal segment C to prevent kinking at this joint. The core wire 42 preferably extends into or just past the transition from the distal segment B to the balloon segment A to prevent kinking adjacent this joint. Any number of spokes 40 can be used.

FIG. 5 illustrates an alternative embodiment of a distal segment B with a kink resisting member in the form of a cross shaped mechanical stop 50. The size, shape, and material of the mechanical stop 50 can be varied to achieve the desired kink resistance, pushability, and flexibility. For example, an extruded polymer stop 50 may be used which has a largest cross section which is just less than the inner diameter of the distal tube 20c. In another example, a cross shaped or other shaped wire stop 50 may be used which has a largest cross section which is about V₂ or less the inner diameter of the distal tube 20c. The mechanical stop 50 can be free floating to improve flexibility of the distal segment B or may be tacked or bonded at one or more locations as discussed above with respect to the core wires. The mechanical stop 50 can be formed in a variety of shapes including cross shape (as shown), triangular, star shaped, or other shapes with any number of outwardly extending portions.

FIG. 6 illustrates an example of a distal segment B which is a combination of the mechanical stop of FIG. 5 and the extrusion of FIG. 4. The FIG. 6 embodiment includes a central core wire 60 onto which a spoke structure 62 is extruded. As in the embodiment of FIG. 4, the spoke height to thickness ratio can be selected to be less than the critical buckling ratio and a metal core wire 60, such as Nitinol can be used to achieve flexibility and pushability. The spoke structure 62 can be formed by extruding can inserted into the tube 20d with a small clearance.

As shown in FIG. 7, the spoke structure 62 of FIG. 6 can be modified by periodically removing material from the spokes with notches 64. The notches 64 can be designed to achieve a desired flexibility and allow fluid transmission between the chambers.

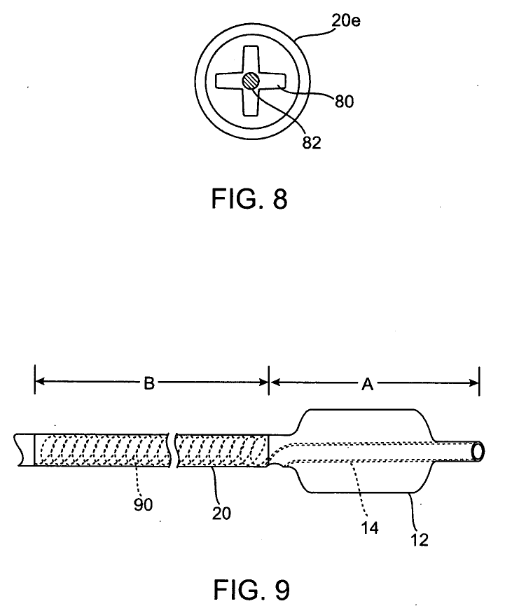

FIG. 8 illustrates a further embodiment of a distal segment B having a mechanical stop member with spokes 80 extruded over a central wire 82 and inserted into a tube 20e. Alternatively, the central wire 82 may be inserted after extrusion of the spokes 80. In the FIG. 8 embodiment, the spokes 80 are tapered in the form of a star shape. Any number of spokes 80 may be used.

FIG. 9 illustrates an alternative embodiment of a kink resistant distal segment B with an inner coil shaped kink resisting member 90. The inner coil 90 may be inside the distal tube, embedded in the distal tube, or partially embedded in the distal tube. As in the previous embodiments, the coil 90 extends to or past the transitions at both ends of the distal segment B to prevent kinking adjacent the transitions. The coil may be varied in wire diameter, pitch, or both along the length of the distal segment B. Although a one wire coil has been illustrated, multiple wire coils or a braided pattern may also be used.

As an alternative to or in addition to the kink resisting members described above, a tapered distal end of the proximal tube 24 of FIG. 1 can be used to further prevent kinking in the distal segment B.

While the invention has been described in detail with reference to the preferred embodiments thereof, it will be apparent to one skilled in the art that various changes and modifications can be made and equivalents employed, without departing from the present invention.

1. A balloon catheter comprising:
a balloon segment having an expandable balloon and a guidewire tube extending through the balloon, the guidewire tube having a proximal port adjacent a proximal end of the balloon;
a distal segment connected to a proximal end of the balloon segment and having an inflation lumen and a kink resisting member extending along the length of the distal segment; and
a proximal segment having flexibility which is less than a flexibility of the distal segment, the proximal segment connected to a proximal end of the distal segment with the kink resisting member extending into or fixed to the proximal segment.

2. The balloon catheter of claim 1, wherein the kink resisting member includes a core wire which is free floating in the inflation lumen.

3. The balloon catheter of claim 2, wherein the kink resisting member is retained at a distal end by a sleeve.

4. The balloon catheter of claim 2, wherein the kink resisting member includes an extruded member over the kink resisting member.

5. The balloon catheter of claim 4, wherein the extruded member is extruded with an outer tube of the distal segment.

6. The balloon catheter of claim 4, wherein the extruded member is inserted into an outer tube of the distal segment.

7. The balloon catheter of claim 1, wherein the balloon segment is fused to the distal segment at a junction area adjacent the proximal port of the guidewire tube.

8. The balloon catheter of claim 7, wherein the guidewire tube is a separate tube from the distal segment and is joined to the balloon and the distal segment at the junction area.

9. A rapid exchange catheter system comprising:
a balloon catheter comprising:
a balloon segment having an expandable balloon and a guidewire tube extending through the balloon, the guidewire tube having a proximal port adjacent a proximal end of the balloon;
a distal segment connected to a proximal end of the balloon segment and having an inflation lumen and a kink resisting member extending along the length of the distal segment; and
a proximal segment having flexibility which is less than a flexibility of the distal segment, the proximal segment connected to a proximal end of the distal segment with the kink resisting member extending into or fixed to the proximal segment wherein the balloon segment is fused to the distal segment at a junction area adjacent the proximal port of the guidewire tube; and
a guidewire slidably positioned in the guidewire tube.

10. The balloon catheter of claim 9, wherein the kink resisting member includes a core wire which is free floating in the inflation lumen.

11. The balloon catheter of claim 10, wherein the kink resisting member is retained at a distal end by a sleeve.

12. The balloon catheter of claim 10, wherein the kink resisting member includes an extruded member over the kink resisting member.

13. The balloon catheter of claim 12, wherein the extruded member is extruded with an outer tube of the distal segment.

14. The balloon catheter of claim 12, wherein the extruded member is inserted into an outer tube of the distal segment.

15. The balloon catheter of claim 9, wherein the guidewire tube is a separate tube from the distal segment and is joined to the balloon and the distal segment at the junction area.
